(19) United States
(12) Patent Application Publication
(10) Pub. No.: US 2016/0374632 A1
(43) Pub. Date: Dec. 29, 2016

(54) RADIATION WINDOW FOR MEDICAL IMAGING SYSTEMS

(71) Applicant: Arineta Ltd., Caesarea (IL)

(72) Inventor: Asaf David, Alonei Abba (IL)

(21) Appl. No.: 14/902,083

(22) PCT Filed: Jul. 8, 2014
(86) PCT No.: PCT/IL14/50614

§ 371 Date: Dec. 30, 2015

Related U.S. Application Data
(60) Provisional application No. 61/844,485, filed on Jul. 10, 2013.

(51) Int. Cl. A61B 6/10; A61B 6/03

(52) U.S. Cl. CPC A61B 6/102; A61B 6/032

(57) ABSTRACT

A radiation window for an X-ray imaging system includes a foam layer sandwiched between a first layer and a second layer of sheet material. The radiation window provides a structural barrier between at least a portion of the X-ray imaging system and an object or patient being imaged.

### FIELD AND BACKGROUND OF THE INVENTION

The present invention, in some embodiments thereof, relates to radiation windows for medical imaging systems and, more particularly, but not exclusively, to a CT scanning window.

Medical imaging system such as X-ray imaging systems, CT scanners, Positron Emission Tomography (PET) imaging systems and Nuclear Medicine (NM), e.g. Single-Photon Emission Computed Tomography (SPECT) imaging systems are known to generate image data from radiation attenuated by a patient or an object for imaging.

X-ray imaging systems typically include an X-ray source for generating X-ray beams, an image detection unit for capturing the X-ray beams after being attenuated by a patient or object to be imaged and accompanying circuitry for powering and controlling the system. Typically, the image detection unit is confined within a housing that includes a radiation window through which X-ray beams are received after attenuation by the patient or object. Often the patient or object is positioned in close proximity to the detection unit during imaging. The housing with radiation window provides a protective barrier that separates the patient, operator and/or object for examination from potentially fragile elements of the image detection unit and also protects the image detection unit against environmental hazards such as dust and fluids.

In CT scanners, an X-ray source and image detection unit is confined within a gantry. The gantry typically includes a cylindrical shaped radiation and/or scan window that defines a bore through which a patient or object is positioned for imaging. During scanning, the X-ray source and the image detection unit typically revolve at high speed around the bore and at close proximity to the patient or object. The X-ray source and image detection unit are positioned in the gantry so that X-ray beams emitted by the X-ray source traverse the radiation window, penetrate through the patient or object for imaging and then traverse the radiation window again before impinging on a detector of the image detection unit. Since the X-ray source and the image detection unit typically revolve at high speed during scanning, the gantry and radiation window are designed as a structural element that can protect the patient from possible collision and/or impact with moving parts.

Typically it is desired to construct the radiation window from a material and/or with a structure that provides adequate rigidity without significantly attenuating the signal (X-rays beam or other). Low attenuation is of particular interest when imaging patients with a given radiation dose. Any further attenuation of the beam after penetration through the patient reduces image quality for that given radiation dose. CT scanner radiation windows are known to be constructed from a polymer sheet and/or from a plate formed from a composite material, e.g. carbon fiber reinforced polymer. The radiation window can optionally be transparent.

BrightSpeed™ Elite is a CT scanner available by General Electric Healthcare is an exemplary CT scanner including a radiation window constructed from a transparent material. The transparent radiation window provides for projecting line markers through the transparent window and toward the patient. The line markers are used to position the patient with respect to the radiation source and detector of the scanner. Typically, the line markers are projected from light sources that are mounted on a rotating frame of the gantry. The BrightSpeed™ scanner additionally includes a transparent portion in the housing adjacent to the radiation window through which a stationary light source projects a line marker. Typically this light source is adapted to project a line marker that is parallel to the rotation axis of the gantry.

Radiation windows for (PET) imaging systems and for NM and/or SPECT imaging systems are also known. In PET and SPECT, the patient becomes a source of gamma-rays after being injected with a radio-labeled pharmaceutical. PET detectors are typically arranged in a static ring within a gantry, allowing detection of pairs of gamma-rays. SPECT detectors modules on the other hand are arranged into flat detectors and are normally rotated around the patient. Radiation windows for PET imaging systems are known to be constructed from a single piece of silk-screened polycarbonate thermoplastic, e.g. Lexan® manufactured by SABIC Innovative Plastics′. Although PET and NM typically operate in higher range of energies than X-ray imaging modalities, requirements of uniform thickness and minimal attenuation for the radiation window are important in these cases as well.

International Patent Application Publication No. WO20012047366 entitled “Polymer layer on X-ray window,” the contents of which are incorporated herein by reference discloses an X-ray window for an X-ray source that is formed with a plurality of thin film layers stacked together, including a thin film layer and a polymer layer. The thin film layer can be diamond grapheme, diamond like carbon, beryllium, and combinations thereof. It is disclosed that a polymer layer and a boron hydride layer can provide improved gas impenetrability and improved corrosion resistance to the thin film layer and can also potentially sustain higher temperatures without breakdown.

Medical equipment parts, such as stretchers and patient support accessories, have been manufactured from sandwich structures.

Japanese Patent Application No. JP2006035671, entitled “FRP structure,” the contents of which are incorporated herein by reference discloses a fiber reinforced polymer (FRP) structure for X-ray instruments that has high rigidity, lightweight property as well as high X ray transmission and damping performance. It is described that the FRP structure can be used in medical equipment parts which need high radio-translucency, such as cassette for radiography, an X-ray picture conversion panel, and CT table-top plate. The FRP structure includes a thermoplastic resin foam layer [A]; an FRP layer [B] having a continuous carbon fiber as a reinforced fiber; and a sheet-like resin layer [C]. The FRP structure has a laminated composition having the structural element [C] with a thickness of 5-200 μm provided on one side of the structural element [B] and the structural element [A] provided on the other side, and a neutral plane of the FRP structure is in the inside of [A].

Japanese Patent Application No. 2002303696, entitled “Radiation Image Conversion Panel,” the contents of which are incorporated herein by reference is referred to in the Japanese Patent Application No. JP2006035671. A radiation image conversion panel that improves the horizontality of a radiation image conversion panel and obtains images of good image quality is proposed. It is disclosed that a stimulable phosphor layer is stacked on a first stiff layer, a filler layer is placed on the side where the stimulable phosphor layer is laminated by being bonded and a second stiff layer is also stacked on the filler layer. In this case, the density of the filler layer shall be lower than those of the first stiff layer and the second stiff layer.

Japanese Patent Application No. JP2008000247, entitled “Top plate for X-ray imaging apparatus,” the contents of which are incorporated herein by reference discloses a top plate for supporting a patient with potentially heavy weight during imaging with an X-ray imaging apparatus that provides improved rigidity while suppressing an increase in X-ray absorption. A cradle includes a core material made of resin foam covered with an exterior including two or more carbon fiber reinforced polymer (CFRP) layers. It is described that a resin film is inserted and laminated between the CFPR layers at a bottom surface part of the exterior where the weight of the cradle and patient is supported but not at the top surface part. Consequently, the number of the CFRP layers required for obtaining desired rigidity is reduced to reduce costs. Moreover, by eliminating the resin film on the upper surface, the X-ray absorption is reduced compared with a structure of inserting a resin film between the CFRP layers of the upper surface.

### SUMMARY OF THE INVENTION

According to an aspect of some embodiments of the present invention there is provided a radiation window that provides for a low attenuation passage for radiation while also operating as a structural barrier between a patient being imaged and the inner parts of the imaging system. According to some embodiments of the present invention, the radiation window has a sandwich construction including a foam core.

According to an aspect of some embodiments of the present invention there is provided a radiation window for an X-ray imaging system including a foam layer sandwiched between a first layer and a second layer of sheet material, wherein the radiation window provides a structural barrier between at least a portion of the X-ray imaging system and an object or patient being imaged.

Optionally, the foam layer includes 2-5 mm layer of thermoplastic resin foam.

Optionally, the foam layer is formed with a foam density in the range of 0.05-0.25 g/cc.

Optionally, each of the first and second layers is formed from at least one of a polymer material, a fiber reinforced polymer composite material and a carbon fiber reinforced polymer composite material.

Optionally, at least one of the first and second layers is formed from a polymer material.

Optionally, the radiation window is a structural barrier between a detector array of an X-ray imaging system and a patient being imaged by the X-ray imaging system.

Optionally, the radiation window is supported by a frame and wherein the frame at least partially surrounds the radiation window.

Optionally, the radiation window is a scan window for a CT scanner.

Optionally, the radiation window is sized for use with a positron emission tomography imaging system or a single-photon emission computed tomography imaging system.

Optionally, the radiation window is sized for use with one of a digital radiography, a film radiography, a computed radiography, a fluoroscopy and an angiography imaging system.

Optionally, at least a portion of the first and second layers are light transparent.

Optionally, the radiation window includes one or more openings across the foam layer, wherein the one or more openings are adapted for radiating light therethrough.

According to an aspect of some embodiments of the present invention there is provided an X-ray imaging system including a housing enclosing: an X-ray source for generating an X-ray beam, and a detector for detecting the X-ray beam as attenuated by an object or patient being imaged, wherein the housing includes a radiation window through which the X-ray beam is received by the detector, the radiation window formed with a foam layer sandwiched between a first layer and a second layer of sheet material and operative to provide a structural barrier between the detector and the patient or object being imaged.

Optionally, the foam layer includes 2-5 mm layer of thermoplastic resin foam.

Optionally, each of the first and second layers is formed from at least one of a polymer material, a fiber reinforced polymer composite material and a carbon fiber reinforced polymer composite material.

Optionally, the radiation window provides a structural barrier between moving parts of the X-ray imaging system and a patient being imaged by the X-ray imaging system.

Optionally, the radiation window is supported by a frame and wherein the frame at least partially surrounds the radiation window.

Optionally, the imaging system is a CT scanner.

Optionally, the imaging system is any one of digital radiography, film radiography, computed radiography, fluoroscopy and angiography imaging system.

Optionally, at least a portion of the first and second layers are light transparent.

Optionally, the radiation window includes one or more openings across the foam layer adapted for radiating light therethrough.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D show an exemplary prior art radiation windows of a flat panel X-ray imaging system, a CT scanner, a PET system and a SPECT imaging system respectively;

FIG. 2 is simplified schematic drawing showing a layered structure of a radiation window in accordance with some embodiments of the present invention;

FIG. 3 is a simplified schematic drawing showing cross-section of a radiation window for a CT scanner in accordance with some embodiments of the present invention; and

FIG. 4 is a radiation window for a CT scanner including light transparent portions in accordance with some embodiments of the present invention.

### DESCRIPTION OF SPECIFIC EMBODIMENTS OF THE INVENTION

The present invention, in some embodiments thereof, relates to radiation windows for medical imaging systems and, more particularly, but not exclusively, to a CT scanning window.

As used herein the term radiation window refers to a portion of a housing through which radiation that is attenuated by a patient or an object to be imaged traverses before reaching an image detection unit that is confined within the housing. Optionally, the radiation window additionally covers an area through which a radiation source of the medical imaging system directs radiation toward the patient or object to be imaged.

Typically, during medical imaging, a patient is required to be close to an image detection unit of the system. Proximity to the image detection unit is known to improve the image quality obtained from the image detection unit and/or reduce the size and cost of the unit. Such proximity to the image detection unit can potentially lead to accidental collision with the image detection unit. Typically, a radiation window is used as a structural barrier for physically separating the patient from the image detection unit without significantly attenuating the radiation to be detected for imaging. For imaging systems that include high speed scanning, e.g. CT scanners, additional mechanical rigidity is typically desired to protect a patient from possible collision with elements in the gantry that rotate around the patient at high speeds.

In known CT scanners that scan with relatively narrow beams, the radiation window is typically constructed from a 0.3-0.5 mm thick polymer sheet. These radiation windows are cylindrical in shape and have a width of 1-4 cm (respective the rotation axis of the scanner) and diameters of 60 cm and above. The 0.3-0.5 mm thick polymer sheets were found to provide a low attenuation barrier with adequate structural rigidity against collision for a cylindrical shaped radiation window of a width ranging from 1-4 cm. The structural rigidity of the radiation window is typically important for preventing any deformation of the radiation window in response to pressure accidently applied on the radiation window by the patient during scanning. Deformation can potentially cause collision with high speed moving parts of the gantry that can damage the CT scanner and also be dangerous to the patient.

CT scanners that scan with a wider beam use a wider radiation window and require a thicker polymer sheet to provide adequate structural rigidity. Recently, CT scanners including larger detector arrays, larger area detectors and/or relatively large arrays of X-ray sources have been developed. In these systems the required width of the cylindrical radiation window can reach to 30 cm or more. To accommodate the wider radiation window and maintain the required rigidity, thicker polymer or composite material sheets of up to 2.5 mm or more are typically used. In other large area radiography systems, a radiation window is constructed from a plate formed of carbon fiber based composite material of 1-2 mm thickness.

Although increasing the thickness of the polymer sheet improves the strength, it also increases the attenuation of the X-ray beam penetrating through the sheet. As a result, image quality is sacrificed and/or higher radiation dose is required to be delivered to the patient in order to preserve image quality. The present inventor has found a method for improving the structural aspects of the radiation window without increasing attenuation of the penetrating X-ray beam, gamma beam or the like.

According to some embodiments of the present invention, the radiation window is constructed with two thin layers of sheet material that are separated by a foam interior, e.g. foam layer. The present inventor has found that by adding the foam layer to the radiation window, the compressive strength of the radiation window against locally applied compression, e.g. due to a patient accidently pushing against the radiation window and/or the protection provided by the radiation window, can be improved without significantly increasing the attenuation of the X-ray beam. The present inventor has found that compared to a solid sheet the sandwiched structure proposed herein is associated with high ratio between structural stiffness and material density. This ratio provides constructing a structurally strong, e.g. rigid radiation window with relatively low X-ray attenuation.

Typically, the foam has very low radiation absorbency. According to some embodiments of the present invention, the foam layer thickness is determined according to specific requirements and may vary significantly depending on the size of the radiation window and the system parameters. Typical foam layer thickness maybe 2-5 mm. Exemplary foam that can be used in accordance with some embodiments of the present invention include s polyurethane with density of 0.22 g/cc, Rohacell® with density of 0.0521 gr/cc available by Severn Valley Sailplanes in the UK and/or PolyVinyl Chloride (PVC) with density of 0.13 gr/cc is used for the foam layer.

In some exemplary embodiments, the external sheet layers are formed with polycarbonate thermoplastic, e.g. Lexan® manufactured by SABIC Innovative Plastics′. In some exemplary embodiments, the external layers are formed with FRP and/or CFRP. Optionally, the external sheet layers are between 0.05-0.3 mm thick depending on the size of the radiation window and the system requirements, however lower or higher thicknesses can be used. According to some embodiments of the present invention, the radiation frame is adapted for use with any one of a digital radiography, a film radiography, a computed radiography, a fluoroscopy and an angiography imaging system.

According to some embodiments of the present invention, the radiation window is held within a frame that at least partially surrounds the radiation window. Optionally for a cylindrical radiation window, the frame is in the form of two rings that support the radiation window from opposite ends. Optionally, for rectangular shaped radiation windows a rectangular shaped frame is used. Optionally the frame is made of one or more of polymer, metal, FRP and CFRP. Optionally, the frame is designed provides isolation any humidity associated with the foam layer and to be a convenient interface for connecting the radiation window to the rest of the gantry or housing.

The present inventor has also found that radiation window as described herein can be used as acoustic barrier to reduce noise generated inside the gantry.

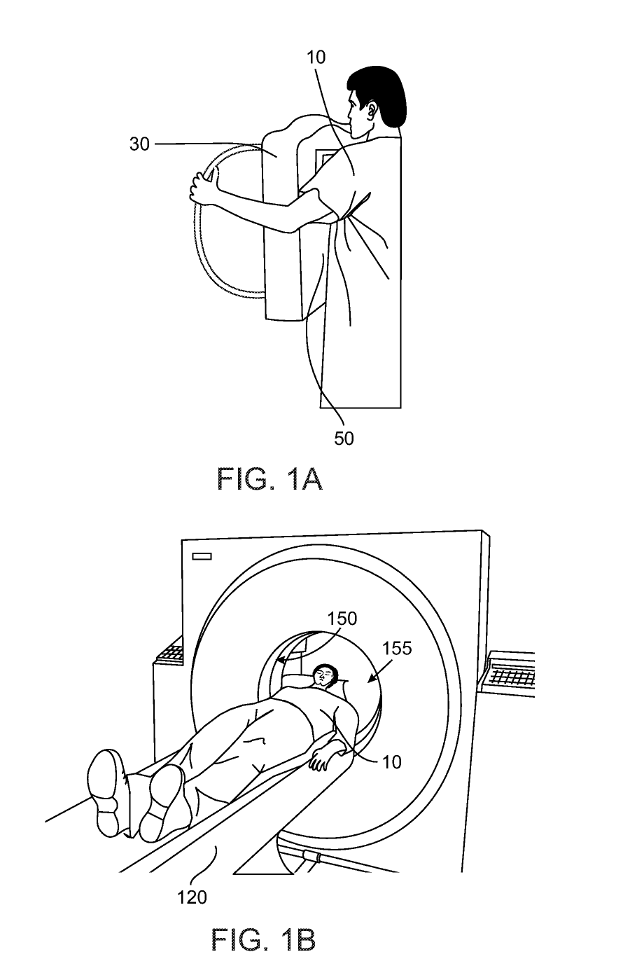

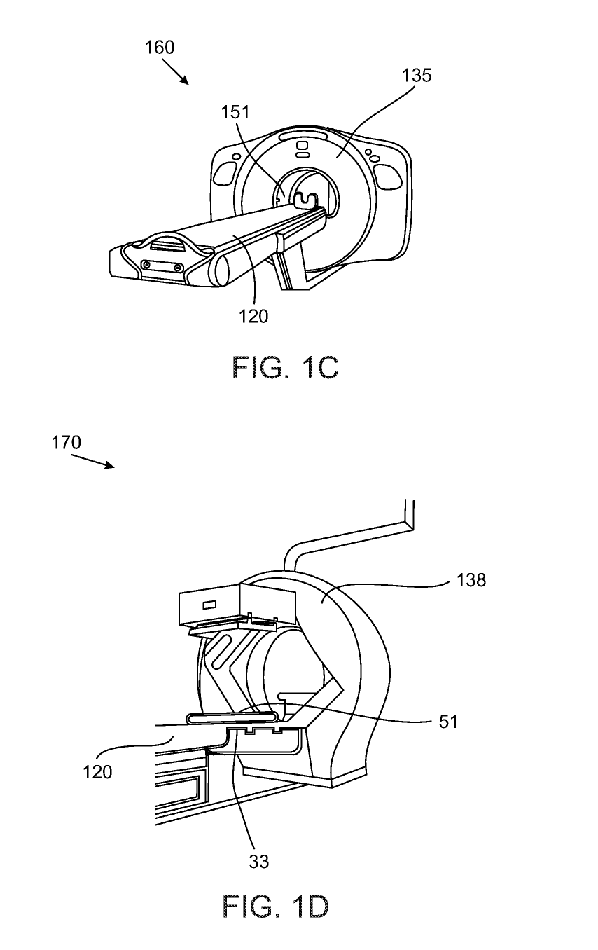

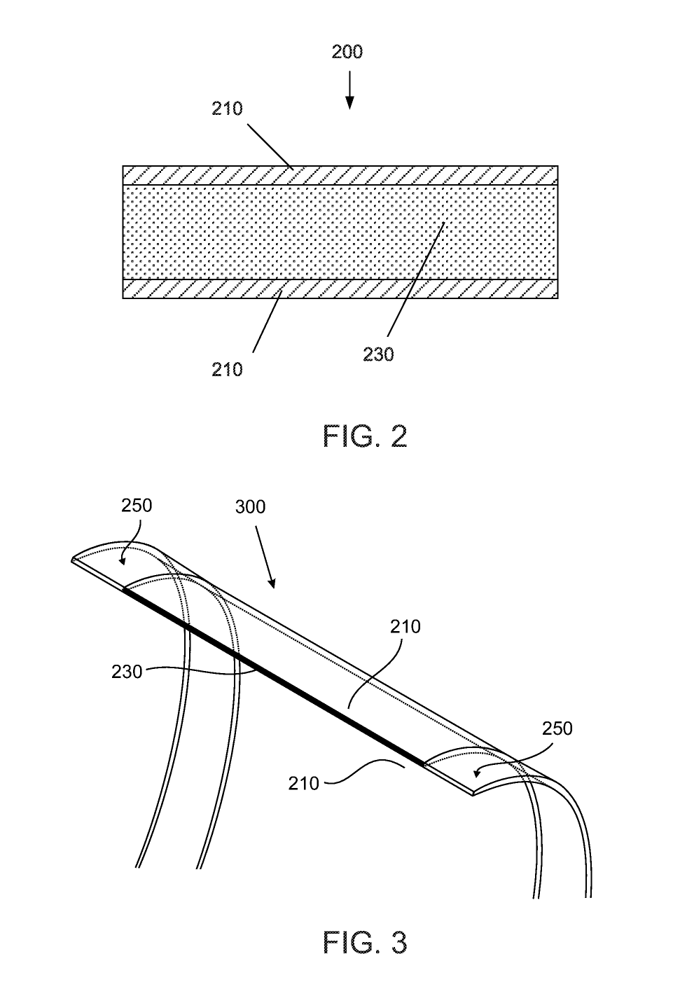

For purposes of better understanding some embodiments of the present invention, as illustrated in FIGS. 2-3 of the drawings, reference is first made to FIG. 1A-1D showing exemplary prior art radiation windows. Referring now to FIG. 1, in known flat panel X-ray radiography systems, a flat radiation window 50 of detector 30 is occasionally brought up against a portion of the patient 10 to be imaged. Occasionally, radiation window 50 is brought into physical contact with patient 10 during the imaging. Typically, a size of radiation window 50 corresponds to a size of a detector array within detector 30 plus an additional margin. Typically, the detector array within detector 30 is operable to receive an X-ray radiation as attenuated by patient 10 and output from the detector array is used to construct an image. Flat panel radiography system is shown by way of example but radiation windows are used in a similar manner in other projection X-Ray imaging systems such as film radiography, computed radiography, fluoroscopy and angiography C arms and others.

FIG. 1B shows a typical CT scanner. In known CT scanners 100, a patient 10 typically lies down on a movable support platform 120 used to move patient 10 in and out of gantry 130. A radiation window 150 typically has a cylindrical shape that defines and/or surrounds a bore 155 in gantry 130 through which patient 10 is positioned for imaging. Typically, gantry 130 houses an X-ray source and detector that rotate at high speed in close proximity to radiation window 150. Although, patient 10 is not required to be in physical contact with radiation window 150, bore 155 is typically size to provide close proximity between the X-ray source and a detector in gantry 130 and patient 10.

FIG. 1C shows a known PET system. Typically, a PET system 160 includes PET detectors that are typically arranged in a static ring within a gantry 135, allowing detection of pairs of gamma-rays through a cylinder shaped radiation window 151. A movable support platform 120 is used to move a patient in and out of gantry 135 during detection. Exemplary PET radiation window 151 is known to be manufactured from a single piece of silk-screened Lexan® that is notched and flanged at one end, and held in place by two pieces of rubber channel.

FIG. 1D shows a known SPECT system. Typically, a SPECT system 170 includes SPECT detectors modules 33 arranged into flat detectors that extend from a gantry 138 and are normally rotated around a patient supported on a support platform 120. Typically, SPECT detectors modules 33 include a radiation window 51 through which gamma rays are received by the flat detectors.

Reference is now made to FIG. 2 showing simplified schematic drawing of a layered structure of a radiation window in accordance with some embodiments of the present invention. According to some embodiments of the present invention, a radiation window 200 is constructed from two layers 210 of sheet material and a third inner layer 230 of foam. It is noted that FIG. 2 shows a cross sectional view of the radiation window 200. According to some embodiments of the present invention, layers 210 are formed with one or more of a polymer material, FRP material and a CFRP. Optionally, layer 210 is formed from Lexan® or other polycarbonates. In some exemplary embodiments, layers 210 are formed from a same material. Alternatively, different materials are used for the different layers. Optionally, each of layers 210 has a thickness that is less than 0.3 mm, e.g. 0.05-0.3 mm. Optionally, thinner layers may be used as long as they provide a required stiffness and/or strength. Typically, the foam layer 230 is significantly thicker than each of layers 210. Optionally, foam layer 230 of between 1-7 mm, e.g. 2-5 mm thick is used. Optionally, layer 210 that is exposed may be painted for aesthetical purposes. Typically, foam layer 230 is associated with a low density, e.g. lower density than layer 210 and therefore provides a relatively low radiation absorption layer. In some exemplary embodiments, radiation window 200 is used to cover a detector array for detecting X-rays attenuated by a patient or object to be images. Optionally radiation window 200 is used as a radiation window for one or more of a flat detector, a CT scanner, a PET system and a SPECT system. In some exemplary embodiments, radiation window 200 is used to cover both the x-ray source, e.g. one or more x-ray sources and the detector array. In some embodiments, radiation window 200 is flat along its length. In some embodiments radiation window 200 is curved in one or more dimensions.

Reference is now made to FIG. 3 showing a simplified schematic cross-section of a radiation window for a CT scanner in accordance with some embodiments of the present invention. Typically, a radiation window 300 for a CT scanner has a generally cylindrical shape. According to some embodiments of the present invention, radiation window is has a sandwich construction including a foam layer 230 sandwiched between two layers 210. Layers 210 and 230 may be similar to layers 210 and 230 described herein in reference to FIG. 2. According to some embodiments of the present invention, radiation window 300 is structurally supported with a frame 250. Optionally, two frames 250 positioned on either side of radiation window 300 are used to support radiation window 300. Optionally, radiation window 300 and frame 250 is provided as a single part that can be easily mounted on and off a CT scanner. Optionally, frame 250 is ring shaped frame that is operable to clasp scanning window 300. Optionally frame 250 is glued to radiation window 300. Optionally frame 250 is made of one or more of polymer, metal, FRP, CFRP. Optionally, radiation window 300 is used for a PET system.

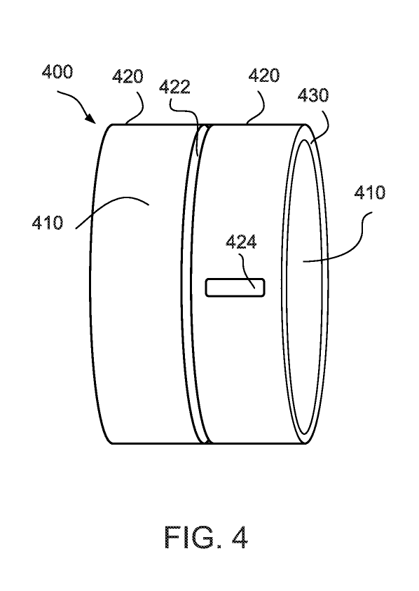

Reference is now made to FIG. 4 showing a radiation window for a CT scanner including transparent portions in accordance with some embodiments of the present invention. According to exemplary embodiments of the present invention, a radiation window 400 is cylindrically shaped radiation window with a sandwich construction including foam 430 sandwiched between two external layers 410. Optionally, external layers 410 are constructed from a transparent material, e.g., a transparent polymer sheet. According to some embodiments of the present invention, radiation window 400 includes a ring shaped light transparent strip 422 through which one or more lines of light and/or line markers that are perpendicular to a rotation axis of the CT scanner are transmitted and/or radiated. In some exemplary embodiments, radiation window 400 additionally includes a transparent strip 424 through which a line of light parallel to the rotation axis of the CT scanner is transmitted and/or radiated. Typically, the line markers radiated through light transmitting strips 422 and 424 are used to position a patient for imaging.

According to some embodiments of the present invention, light transparent strips 422 and 424 include external layers 410 that are transparent to light but do not include foam 430. Optionally, transparent strips 422 and/or 424 are formed by introducing openings in the foam layer and/or excluding the foam layer in the area designated as the transparent strip. Optionally, transparent strips 422 and 424 only include one external layer 410, e.g. the external layer closest to the patient. Optionally, radiation window 400 includes opaque material for external layers 410 in areas 420 and includes light transmitting material in an area of strips 422 and 424. Alternatively and/or additionally, a frame connecting radiation window 400 to a gantry is fully or partially formed with a light transparent material and line markers for positioning the patient is projected through the frame.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

1. A radiation window for an X-ray imaging system comprising a foam layer sandwiched between a first layer and a second layer of sheet material, wherein the radiation window provides a structural barrier between at least a portion of the X-ray imaging system and an object or patient being imaged.

2. The radiation window according to claim 1, wherein the foam layer includes 2-5 mm layer of thermoplastic resin foam.

3. The radiation window according to claim 1, wherein the foam layer is formed with a foam density in the range of 0.05-0.25 g/cc.

4. The radiation window according to claim 1, wherein each of the first and second layers is formed from at least one of a polymer material, a fiber reinforced polymer composite material and a carbon fiber reinforced polymer composite material.

5. The radiation window according to claim 1, wherein at least one of the first and second layers is formed from a polymer material.

6. The radiation window according to claim 1, wherein the radiation window is a structural barrier between a detector array of an imaging system and a patient being imaged by the imaging system.

7. The radiation window according to claim 1, wherein the radiation window is supported by a frame and wherein the frame at least partially surrounds the radiation window.

8. The radiation window according to claim 1, wherein the radiation window is a scan window for a CT scanner.

9. The radiation window according to claim 1, wherein the radiation window is part of a positron emission tomography imaging system or a single-photon emission computed tomography imaging system.

10. The radiation window according to claim 1, wherein the radiation window is part of a digital radiography, a film radiography, a computed radiography, a fluoroscopy or an angiography imaging system.

11. The radiation window according to claim 1, wherein at least a portion of the first and second layers are light transparent.

12. The radiation window according to claim 11, wherein the radiation window includes one or more openings across the foam layer, wherein the one or more openings are adapted for radiating light therethrough.

13. An X-ray imaging system comprising:
a housing enclosing:
an X-ray source for generating an X-ray beam; and
a detector for detecting the X-ray beam as attenuated by an object or patient being imaged;
wherein the housing includes a radiation window through which the X-ray beam is received by the detector,
the radiation window formed with a foam layer sandwiched between a first layer and a second layer of sheet material and operative to provide a structural barrier between the detector and the patient or object being imaged.

14. The X-ray imaging system according to claim 13, wherein the foam layer includes 2-5 mm layer of thermoplastic resin foam.

15. The X-ray imaging system according to claim 13, wherein each of the first and second layers is formed from at least one of a polymer material, a fiber reinforced polymer composite material and a carbon fiber reinforced polymer composite material.

16. The X-ray imaging system according to claim 13, wherein the radiation window provides a structural barrier between moving parts of the X-ray imaging system and a patient being imaged by the X-ray imaging system.

17. The X-ray imaging system according to claim 13, wherein the radiation window is supported by a frame and wherein the frame at least partially surrounds the radiation window.

18. The X-ray imaging system according to claim 13, wherein the imaging system is a CT scanner.

19. The X-ray imaging system according to claim 13, wherein the imaging system is any one of digital radiography, film radiography, computed radiography, fluoroscopy and angiography imaging system.

20. The X-ray imaging system according to any one claim 13, wherein at least a portion of the first and second layers are light transparent.

21. The X-ray imaging system according to claim 20, wherein the radiation window includes one or more openings across the foam layer adapted for radiating light therethrough.
